(19) United States
(12) Patent Application Publication
(10) Pub. No.: US 2021/0161433 A1
(43) Pub. Date: Jun. 3, 2021

(54) Sensor Device and Method of Manufacturing a Sensor Device

(71) Applicant: OSRAM OLED GmbH, Regensburg (DE)

(72) Inventors: Martin Brandl, Kelheim (DE); Zeljko Pajkic, Regensburg (DE); Luca Haiberger, Regensburg (DE)

(21) Appl. No.: 17/262,647

(22) PCT Filed: Jul. 23, 2019
(86) PCT No.: PCT/EP2019/069808

§ 371 Date: Jan. 22, 2021

(30) Foreign Application Priority Data
Jul. 26, 2018 (DE) 10 2018 118 110.8

(51) Int. Cl. A61B 5/1455; G01N 33/487; A61B 5/00

(52) U.S. Cl. CPC A61B 5/1455; A61B 5/14517; A61B 5/681; G01N 33/487

(57) ABSTRACT

In an embodiment a portable electronic device includes a sensor device including at least one light emitter, at least one light detector, a housing in which the at least one light emitter and the at least one light detector are arranged and at least one channel forming a passageway through the housing, wherein the at least one light emitter and the at least one light detector are arranged such that light emitted from the at least one light emitter passes through the at least one channel and is thereafter detected by the at least one light detector.

This patent application is a national phase filing under section 371 of PCT/EP2019/069808, filed Jul. 23, 2019, which claims the priority of German patent application 10 2018 118 110.8, filed Jul. 26, 2018, each of which is incorporated herein by reference in its entirety.

### TECHNICAL FIELD

The present invention relates to a sensor device and a method of manufacturing a sensor device.

### BACKGROUND

Smartwatches that monitor various body functions, such as heart rate or blood oxygen saturation, in addition to activity tracking, are becoming increasingly popular.

In addition to monitoring heart rate, which is done either via a pulse belt or, in the case of modern watches, via optical heart rate measurement, it is desirable to analyze body fluids, especially sweat. By analyzing the sweat contents, statements can be made, for example, regarding the athlete's fitness level, e.g. by means of a lactate analysis, the need for mineral intake, e.g. by means of an electrolyte analysis, or even possible diseases.

### SUMMARY

Embodiments provide a sensor device that is suitable for use in the analysis of body fluids and, in particular, can be integrated into a smartwatch. Further embodiments provide a method for manufacturing a sensor device.

A sensor device comprises at least one light emitter configured to emit light and at least one light detector configured to detect light. The at least one light emitter and the at least one light detector are arranged in a housing. At least one channel extends through the housing. The at least one channel may be configured to receive body fluids. The at least one channel may have a diameter such that a fluid, in particular a body fluid such as sweat, may be drawn into the at least one channel by means of a capillary effect. For example, the at least one channel may have a diameter of at most 1 mm. However, the diameter may also be larger or smaller.

The at least one channel forms a through hole or passageway through the housing, i.e., the at least one channel extends from a first outer surface of the housing to a second outer surface of the housing, which may be opposite the first outer surface, for example. Accordingly, a liquid or gas entering the at least one channel at the first outer surface of the housing may exit the at least one channel at the second outer surface of the housing. Consequently, the sensor device is configured such that body fluid can be received into the at least one channel when the sensor device is suitably placed on the skin of a person, without the need for further devices, in particular pumps or the like.

In the sensor device, the at least one light emitter and the at least one light detector are arranged in such a way that the light emitted by the at least one light emitter at least partially first passes through the at least one channel and is then detected by the at least one light detector.

The light emitted by the at least one light emitter may pass through the at least one channel in any chosen direction, for example in a direction perpendicular or approximately perpendicular to the direction of propagation of the at least one channel, i.e., perpendicular to the direction of flow of the liquid or gas in the at least one channel. Furthermore, the light emitted by the at least one light emitter may also pass through the at least one channel, at least partially, multiple times, for example by means of one or more light reflecting surface(s), before the light impinges on the at least one light detector.

The light detected by the at least one light detector can be analysed using any analysis method known to the skilled person. For example, spectroscopic analysis methods are familiar to the skilled person. When light shines through or passes through the at least one channel, the light can be at least partially absorbed by the liquid or gas contained in the at least one channel. It is also possible that only light of certain wavelengths is absorbed. Based on the spectrum of the light emitted by the at least one light emitter and the spectrum of the light detected by the at least one light detector, conclusions can be drawn about the substances present in the at least one channel at that time. In particular, the concentration of certain substances can be determined. For example, the sensor device can be used to obtain data on mineral(s), lactate molecules and/or blood sugar (glucose) contained in the fluid.

The light emitted by the at least one light emitter or the light detected by the at least one light detector may be, for example, light in the visible range, ultraviolet (UV) light, and/or infrared (IR) light.

The at least one light emitter and/or the at least one light detector can be optoelectronic semiconductor components, in particular semiconductor chips. For example, a light emitter can be designed as a light-emitting diode (LED), as an organic light-emitting diode (OLED), as a light-emitting transistor or as an organic light-emitting transistor. Furthermore, an LED, an OLED or a correspondingly designed, in particular organic transistor, can also be designed as a light detector. The at least one light emitter and/or the at least one light detector can furthermore be part of an integrated circuit.

In addition to the at least one light emitter and/or the at least one light detector, other semiconductor devices and/or other components may be integrated into the sensor device.

The sensor device can be manufactured relatively inexpensively and also very compactly. This allows the sensor device to be used in consumer products, also called consumer goods or consumer products, and in particular in wearable electronic devices, such as a smartwatch.

The sensor device, in particular the housing of the sensor device, may have a size, i.e. extension, of at most 10 mm in a first dimension. Also in a second dimension, the sensor device may have a size of at most 10 mm. In a third dimension, the sensor device may have a size of at most 3 mm. The three dimensions may each be orthogonal to each other and be described, for example, by the x, y and z axes of a Cartesian coordinate system.

In one embodiment, the sensor device comprises a substrate having at least one opening, which is in particular a through hole. The at least one light emitter and the at least one light detector are mounted on the substrate, and the at least one channel extends through the at least one opening. In particular, the at least one channel is arranged between the at least one light emitter and the at least one light detector.

The substrate can, for example, be a lead frame that is overloaded with a mold compound, in particular a plastic. Furthermore, the substrate can be a so-called QFN (quad flat no leads package) flat old. A QFN flat old consists of a lead frame, in particular a coated copper lead frame, which is overloaded by a mold compound, wherein the mold compound has the same height as the lead frame, i.e., no cavities are created. The at least one opening may extend through the lead frame and/or the mold compound. The substrate may further be a printed circuit board (PCB), a ceramic substrate, or any other suitable substrate.

A transparent body may be mounted on the at least one opening of the substrate. The transparent body includes at least one passageway, i.e., a channel. The at least one passageway forms a portion of the at least one channel. For example, the transparent body may be mounted on the substrate above the at least one opening and may not extend into the at least one opening. In this case, the at least one opening in the substrate may form the at least one channel together with the at least one passageway in the transparent body. In particular, a liquid may be drawn into the at least one channel by a capillary effect. For example, the transparent body may be a glass capillary.

Transparent in this context means that the body is at least transparent to at least part of the light emitted by the at least one light emitter or at least to light in a certain wavelength range, such that the light of this wavelength range is absorbed as little as possible by the body itself.

As an alternative to a body placed on the substrate, a transparent body may be inserted into the at least one opening in the substrate. The body has a plurality of microchannels forming at least a portion of the at least one channel or forming the entire at least one channel. The microchannels provide an enhanced capillary effect to draw fluid into the microchannels. The microchannels may each comprise a diameter in the range of 1 μm to 1,000 μm, and in particular in the range of 200 m to 300 μm.

The at least one light emitter and the at least one light detector may be encapsulated with a transparent material. Again, transparent means that the material is at least transparent to at least a portion of the light emitted by the at least one light emitter or at least to light in a particular wavelength range. For example, the transparent material may be a transparent silicone.

A light-reflecting or reflective material can be applied to the transparent material. Reflective here means that the material is at least reflective to at least a portion of the light emitted by the at least one light emitter, or at least to light in a particular wavelength range. The light reflective material may have the function of guiding the light generated by the at least one light emitter to the at least one channel with as little loss as possible to pass through the liquid or gas in the at least one channel, and then guiding the light to the at least one light detector with as little loss as possible. For example, the reflective material may be a silicone with TiO₂.

The interface between the transparent material in which the light is guided and the reflective material adjacent to the transparent material may have a particular shape that allows light emitted from the at least one light emitter to be guided to the at least one light detector. For example, the shape of at least a portion of the interface may be like a parable or parabolic.

The transparent material and the light reflective material may encapsulate the at least one light emitter and the at least one light detector, and may form at least a portion of a housing.

Instead of passing the liquid or gas to be analysed through a body having at least one passageway or a plurality of microchannels, such a body can be dispensed with and the channel can be formed by the opening in the substrate, the transparent material, and the light reflective material. In this case, the sidewalls of the at least one channel are formed at least partly by the transparent material and the light reflective material.

According to a further embodiment, the sensor device comprises a substrate on which the at least one light emitter and the at least one light detector are mounted. The substrate may be configured as the substrate described above, but doesn't need to comprise an opening. The at least one channel extends above the at least one light emitter as well as the at least one light detector. In other words, the at least one light emitter as well as the at least one light detector are arranged between the substrate and the at least one channel. The at least one channel may extend in a direction substantially parallel to a major surface of the substrate. Further, the channel may include a plurality of microchannels, particularly having the embodiments described above. Further, a body having a plurality of microchannels may be arranged on the at least one light emitter as well as the at least one light detector.

A light reflective layer can be arranged above the at least one channel. The light reflective layer can in particular be a mirror. Consequently, the light emitted by the at least one light emitter first passes through the at least one channel, is then reflected by the reflective layer, and passes through the at least one channel in the reverse direction to reach the at least one light detector. An advantage of this embodiment is that the light passes through the at least one channel twice and consequently the absorption by the liquid in the at least one channel is increased accordingly.

To prevent light from traveling directly from the at least one light emitter to the at least one light detector and thus falsifying the measurement results, a light reflective material or a light absorbing material may be applied to the substrate between the at least one light emitter and the at least one light detector.

The sensor device may include an analysis unit that performs the above-described analysis of a liquid and/or gas located in the at least one channel based on light emitted from the at least one light emitter and light detected by the at least one light detector.

The analysis unit may be arranged on the substrate together with the at least one light emitter and the at least one light detector, but may also be arranged on a separate substrate or circuit board. In particular, the analysis unit may be an integrated circuit (IC) and may comprise a data memory.

It can be provided that the sensor device comprises exactly one light emitter and/or exactly one light detector. However, the sensor device may also include multiple light emitters and/or multiple light detectors. In the latter case, at least two of the light emitters may emit light of different wavelengths and/or at least two of the light detectors may detect light of different wavelengths.

The sensor device may be integrated into a wearable electronic device. The wearable electronic device may be a so-called “wearable”, i.e., an electronic device that is attached to the user's body or integrated into the user's clothing, such as an activity or fitness tracker or a smartwatch. A smartwatch (English for “smart watch”) is an electronic wristwatch that has additional sensors, actuators, and/or computer functionality or connectivity. Further, the sensor device may be integrated into a piece of jewellery, such as a finger ring, earring, or necklace. Wearing the device directly on the user's body provides a simple means of collecting body fluids, particularly sweat, into the at least one channel for subsequent analysis. Further, the wearable electronic device may be a handheld device or portable device, such as a smartphone, tablet, or handheld medical device.

The wearable electronic device may include an attachment device connected to the sensor device for attaching the sensor device to a body part of a person. For example, the attachment device may be a wristband or a heart rate belt or chest strap.

The sensor device described in the present application can be used to analyse body fluids, in particular sweat. The analysis of sweat represents a very simple alternative compared to blood glucose testing, via which many similar conclusions, e.g., lactate and blood glucose concentration, are possible.

A method of manufacturing a sensor device comprises providing at least one light emitter and at least one light detector, as well as encapsulating the at least one light emitter and the at least one light detector in a housing. At least one channel forms a passageway through the housing. Further, the at least one light emitter and the at least one light detector are arranged such that light emitted from the at least one light emitter at least partially passes through the at least one channel and is thereafter detected by the at least one light detector.

The method of manufacturing a sensor device may include the sensor device embodiments described above.

### BRIEF DESCRIPTION OF THE DRAWINGS

In the following, embodiments of the invention are explained in more detail with reference to the accompanying drawings.

FIGS. 1A to 1B show illustrations of an embodiment of a sensor device with a channel for receiving body fluids;

FIG. 2 shows an illustration of an embodiment of a method of manufacturing a sensor device;

FIG. 3 shows an illustration of an embodiment of a wearable electronic device with a sensor device;

FIGS. 4A to 4B show illustrations of an embodiment of a sensor device with a plurality of microchannels;

FIG. 5 shows an illustration of an embodiment of a sensor device having a channel in a layer of transparent and reflective material; and

FIG. 6 shows an illustration of an embodiment of a sensor device with a plurality of horizontally running microchannels.

In the following detailed description, reference is made to the accompanying drawings, which form a part of this description and in which specific embodiments in which the invention may be practiced are shown for illustrative purposes. Since components of embodiments may be positioned in a number of different orientations, the directional terminology is for illustrative purposes and is not limiting in any way. It is understood that other embodiments may be used and structural or logical changes may be made without departing from the scope of protection. It is understood that the features of the various embodiments described herein may be combined with each other, unless specifically indicated otherwise. Therefore, the following detailed description is not to be construed in a limiting sense. In the figures, identical or similar elements are provided with identical reference signs where appropriate.

### DETAILED DESCRIPTION OF ILLUSTRATIVE EMBODIMENTS

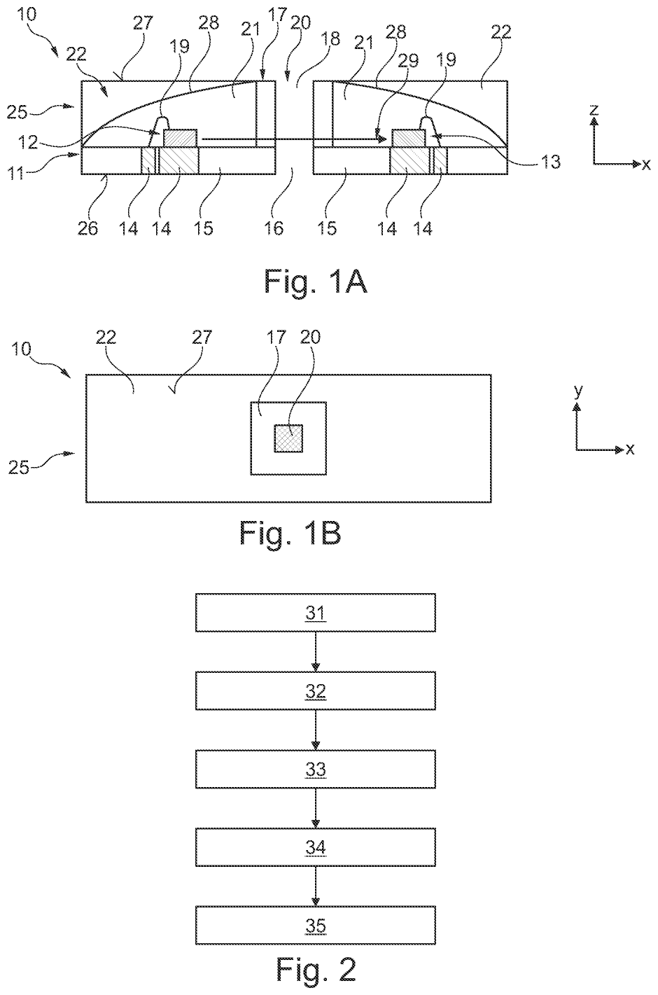

FIG. 1A schematically shows a sensor device 10 in a sectional view. FIG. 1B shows the sensor device 10 in a top view from above.

The sensor device 10 includes a substrate 11 on which a light emitter 12 in the form of an LED semiconductor chip and a light detector 13 also in the form of an LED semiconductor chip are mounted.

In the present embodiment, the substrate 11 is a QFN flatmold comprising a coated copper lead frame 14 that has been overmolded by a mold compound 15. The mold compound 15 has the same height as the lead frame 14, i.e., the top and bottom surfaces of the lead frame 14 are not covered by the mold compound 15. The LED semiconductor chips of the light emitter 12 and the light detector 13 each have an electrode on their bottom side and their top side. The light emitter 12 and the light detector 13 are soldered with their electrode on the bottom side to a respective contact element of the lead frame 14. A bonding wire 19 leads from the electrodes on the upper sides of each of the light emitter 12 and the light detector 13 to a further contact element of the lead frame 14.

The substrate 11 further includes an opening 16 in the form of a recess extending completely through the substrate 11. A body 17 is applied over the opening 16 in the substrate 11, which comprises transparent side walls and through which a passageway 18 extends in the vertical direction. The body 17 may be, for example, a thin glass capillary. The opening 16 in the substrate 11 and the passageway 18 through the body 17 form a channel 20. The channel 20 is arranged between the light emitter 12 and the light detector 13.

The light emitter 12 and the light detector 13 are encapsulated with a transparent material 21, which may be a transparent silicone. A highly reflective material 22 is applied to the transparent material 21, which may be, for example, a silicone mixed with TiO₂ particles. The transparent material 21 and the highly reflective material 22, together with the substrate 11, form a housing 25 in which the light emitter 12 and the light detector 13 are arranged.

The channel 20 forms a passageway through the housing 25, i.e., it extends from a bottom surface 26, i.e., a first outer surface, to a top surface 27, i.e., a second outer surface, of the housing 25.

An interface 28 between the transparent material 21 and the highly reflective material 22 has a predetermined shape. In the sectional view of FIG. 1A, the interface 28 is parabolic.

In addition to the light emitter 12 and the light detector 13, further light emitters and/or light detectors can be mounted on the substrate 11. In particular, the further light emitters or detectors can be designed to generate or detect light of different wavelengths.

The sensor device 10 resp. the housing 25 may have a size in the x-direction shown in FIGS. 1A and 1B of at most 10 mm. In the y-direction, the sensor device 10 resp. the housing 25 may also have a size of at most 10 mm. In the z-direction, the sensor device 10 resp. the housing 25 may have a size of at most 3 mm.

During operation of the sensor device 10, a portion of the light emitted from the light emitter 12 travels directly to the light detector 13. The light thereby passes through the transparent material 21 and the channel 20 located between the light emitter 12 and the light detector 13. Light that is not emitted from the light emitter 12 in a direct direction to the light detector 13 is reflected at the interface 28 by the highly reflective material 22 and is reflected toward the channel 20 due to the shape of the interface 28. It passes through the channel 20 and can be detected by the light detector 13 after any possible further reflection at the interface 28. Consequently, the design of the interface 28 causes a large portion of the light emitted by the light emitter 12 to pass through the channel 20 and subsequently be detected by the light detector 13. In FIG. 1A, the beam path of the light emitted by the light detector 13 is symbolically represented by an arrow 29.

FIG. 2 schematically illustrates a method of manufacturing the sensor device 10 shown in FIGS. 1A and 1B.

In a step 31, the substrate 11 with the opening 16 is provided. The substrate 11 can be pre-produced.

In a step 32, the transparent body 17 is applied to the substrate 11 such that the opening 16 in the substrate 11 and the passageway 18 through the body 17 form the channel 20. The body 17 may, for example, be glued to the substrate 11 or otherwise attached to the substrate 11.

In a step 33, the light emitter 12 and the light detector 13 are soldered to the substrate 11 and the bonding wires 16 are generated.

In a step 34, the light emitter 12 and the light detector 13 are encapsulated with the transparent material 21.

In a step 35, the highly reflective material 22 is applied to the transparent material 21.

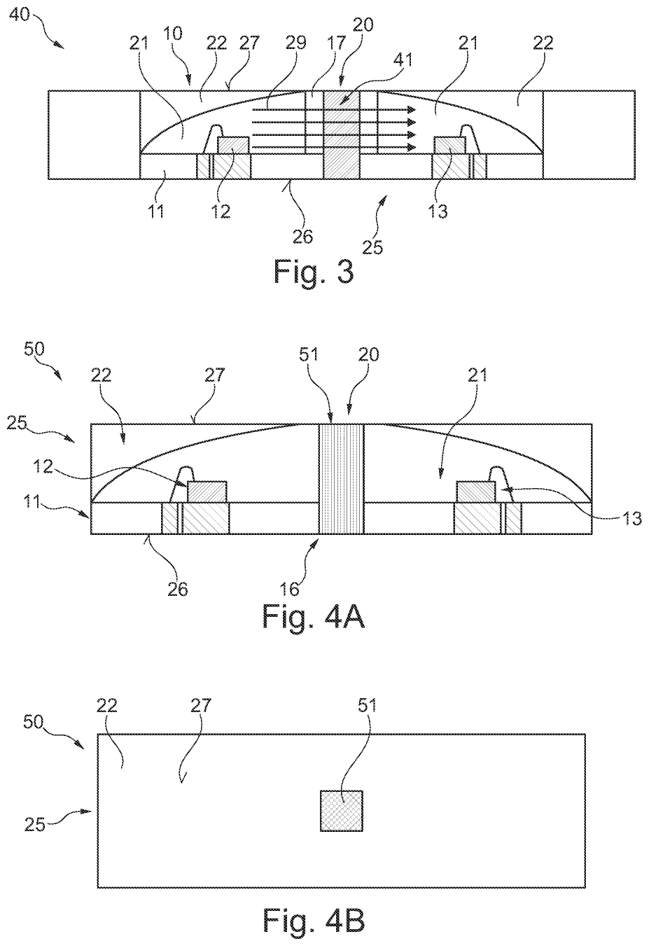

FIG. 3 schematically shows a wearable electronic device 40, for example an activity or fitness tracker or a smartwatch, with the sensor device 10 described above in a sectional view.

The sensor device 10 may be integrated into the device 40 such that the bottom surface 26 and/or the top surface 27 of the housing 25 are exposed. However, it is also conceivable that the bottom surface 26 and/or the top surface 27 are not exposed. In many applications, however, it should be ensured that during operation of the device 40 a surface of the device 40, for example the bottom surface 26 or the top surface 27 of the housing 25, rests on the skin of the user, so that body fluid 41, in particular sweat, enters the channel 20, in particular by a capillary effect, and can be analysed by means of the light emitted by the light emitter 12 and detected by the light detector 13.

For analysing the body fluid 41, the device 40 has an analysis unit not shown in FIG. 3, which may in particular be designed as an integrated circuit. The analysis unit evaluates the light detected by the light detector using methods familiar to those skilled in the art, and can draw conclusions about the fluid 41 therefrom. The analysis unit may be integrated into the sensor device 10 or may be located outside the sensor device 10 in the device 40.

FIG. 4A schematically shows a sensor device 50 in a sectional view. FIG. 4B shows the sensor device 50 in a top view.

The sensor device 50 corresponds in large parts to the sensor device 10 described above. However, unlike the sensor device 10, the sensor device 50 does not include the transparent body 10 with the passageway 18, but a transparent body 51 with a plurality of microchannels forming a plurality of channels 20.

Furthermore, the body 51 is not placed on the opening 16 in the substrate 11, but is inserted into the opening 16. Consequently, the microchannels of the body 51 extend from the bottom 26 to the top 27 of the housing 25.

The microchannels can each have a diameter in the range from 1 μm to 1,000 μm and in particular in the range from 200 m to 300 μm. The advantage of the many thin microchannels is the enhanced capillary effect, through which the body fluid is transported into the microchannels more quickly or more easily.

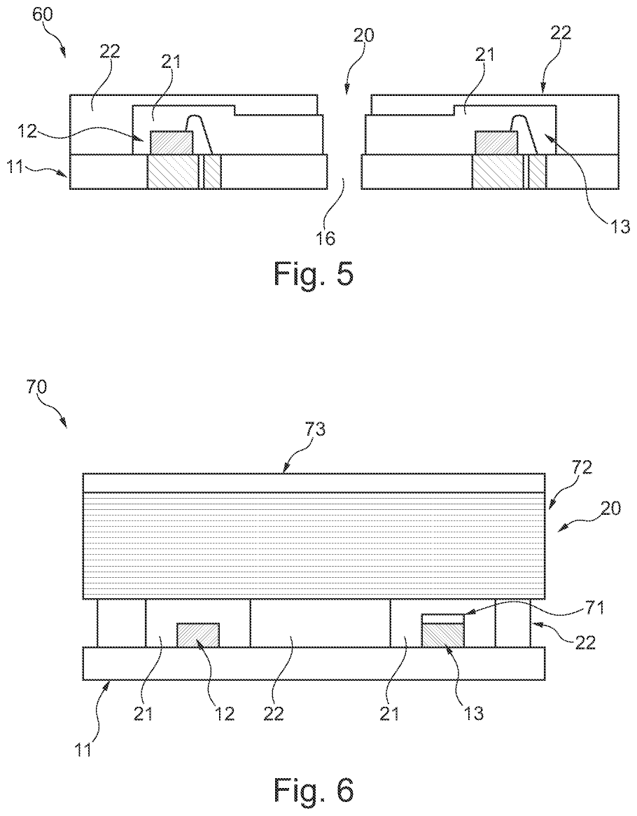

FIG. 5 schematically shows a sensor device 60 in a sectional view similar to sensor devices 10 and 50. However, the sensor device 60 does not include a transparent body with a passageway or a plurality of microchannels.

After the light emitter 12 and the light detector 13 have been applied to the substrate 11, they are overmolded with a transparent material 21, e.g. by transfer molding, also called injection molding. In this process, a recess is kept free in the transparent material 21 above the opening 16 in the substrate 11.

A reflective mold compound is then applied as reflective material 22 in a further transfer molding step. A recess is also kept free in the reflective material 22 above the opening 16 in the substrate 11. The mold compound may contain, for example, an epoxy resin or a silicone.

The recesses in the transparent and reflective materials 21, 22, together with the opening 16 in the substrate 11, form the channel 20 into which the body fluid can be introduced for analysis. Consequently, the side walls of the channel 20 are formed by the substrate 11, the transparent material 21 and the reflective material 22. Thus, the transparent body with a passageway or a plurality of microchannels can be saved.

During operation of the sensing device 60, light emitted from the light emitter 12 reflects off the reflective material 22 and travels in a horizontal direction after passing through the channel 20 to the light detector 13.

FIG. 6 schematically shows a sensor device 70 in a sectional view.

In the sensor device 70, the light emitter 12 and the light detector 13 are mounted on a substrate 11, which may be a QFN flatmold, a printed circuit board, a ceramic substrate, or any other suitable substrate. In the present embodiment, an optional filter 71 is also mounted on the light detector.

After the light emitter 12 and the light detector 13 are applied, both are embedded in a transparent material 21, for example an epoxy resin or a silicone. The space or spaces between the light emitter 12 and the light detector 13 are then filled with a highly reflective material 22 such that there is no line of sight between the light emitter 12 and the light detector 13 and light emitted by the light emitter 12 can only reach the light detector 13 via the liquid to be analysed.

In a subsequent step, a transparent body 72 having a plurality of microchannels is applied to the transparent material 21 and the highly reflective material 22. The microchannels in the body 72 form the plurality of channels 20. The microchannels of the body 72 may be formed similarly to the microchannels of the body 51 described above, but the microchannels in the sensor device 70 extend horizontally, i.e., the microchannels extend parallel to a main surface of the substrate 11. The microchannels may each have a diameter in the range of 1 μm to 1,000 μm, and in particular in the range of 200 μm to 300 μm.

A mirror 73 is then placed on the body 72.

During operation of the sensor device 70, light from the light emitter 12 passes through the fluid in the microchannels of the body 72 and is reflected back via the mirror 73, causing the reflected light to pass to the light detector 13.

Although the invention has been illustrated and described in detail by means of the preferred embodiment examples, the present invention is not restricted by the disclosed examples and other variations may be derived by the skilled person without exceeding the scope of protection of the invention.

1-18. (canceled)

19. A portable electronic device comprising:
a sensor device comprising:
at least one light emitter;
at least one light detector;
a housing in which the at least one light emitter and the at least one light detector are arranged; and
at least one channel forming a passageway through the housing,
wherein the at least one light emitter and the at least one light detector are arranged such that light emitted from the at least one light emitter passes through the at least one channel and is thereafter detected by the at least one light detector.

20. The portable electronic device according to claim 19, wherein the sensor device comprises a size of at most 10 mm in a first dimension and a second dimension, respectively, and a size of at most 3 mm in a third dimension.

21. The portable electronic device according to claim 19,
wherein the sensor device comprises a substrate having at least one opening,
wherein the at least one light emitter and the at least one light detector are mounted on the substrate, and
wherein the at least one channel extends through the at least one opening.

22. The portable electronic device according to claim 21, further comprising a transparent body having at least one passageway forming part of the at least one channel, wherein the transparent body is mounted on the at least one opening of the substrate.

23. The portable electronic device according to claim 21, further comprising a transparent body, wherein a plurality of microchannels of the transparent body is located in the at least one opening of the substrate, and wherein the microchannels form at least a portion of the at least one channel.

24. The portable electronic device according to claim 19, wherein the at least one light emitter and the at least one light detector are encapsulated with a transparent material.

25. The portable electronic device according to claim 24, wherein a light reflective material is applied to the transparent material.

26. The portable electronic device according to claim 25, wherein the at least one channel has sidewalls formed at least in part by the transparent material and the light reflective material.

27. The portable electronic device according to claim 19,
wherein the sensor device comprises a substrate and the at least one light emitter and the at least one light detector are mounted on the substrate, and
wherein the at least one channel extends above the at least one light emitter and the at least one light detector.

28. The portable electronic device according to claim 27, further comprising a light reflective layer arranged above the at least one channel.

29. The portable electronic device according to claim 27, further comprising a light reflective material arranged on the substrate between the at least one light emitter and the at least one light detector.

30. The portable electronic device according to claim 19, wherein the sensor device comprises an analysis unit configured to analyse liquid and/or gas in the at least one channel based on the light detected by the at least one light detector.

31. The portable electronic device according to claim 19,
wherein the sensor device comprises a plurality of light emitters and at least two of the plurality of light emitters are configured to emit light of different wavelengths, and/or
wherein the sensor device comprises a plurality of light detectors and at least two of the plurality of light detectors are configured to detect light of different wavelengths.

32. The portable electronic device according to claim 19, wherein the at least one light emitter and/or the at least one light detector are optoelectronic semiconductor devices.

33. The portable electronic device according to claim 19, further comprising an attachment device connected to the sensor device, wherein the attachment device is a wristband or pulse strap for attaching the sensor device to a body part of a person.

34. The portable electronic device according to claim 19, wherein the portable electronic device is configured to analyse body fluids.

35. A method of manufacturing a sensor device, the method comprising:
providing at least one light emitter and at least one light detector; and
encapsulating the at least one light emitter and the at least one light detector in a housing,
wherein at least one channel forms a passageway through the housing, and
wherein the at least one light emitter and the at least one light detector are arranged such that light emitted from the at least one light emitter passes through the at least one channel and is thereafter detected by the at least one light detector.
